(19) United States
(12) Patent Application Publication
(10) Pub. No.: US 2024/0293610 A1
(43) Pub. Date: Sep. 5, 2024

(54) SYSTEM AND METHOD OF MONITORING AND MANAGING A REMOTE INFUSION REGIMEN

(71) Applicant: ICU Medical, Inc., San Clemente, CA (US)

(72) Inventors: James D. Jacobson, Lindenhurst, IL (US); Shawn Mitchell Folkes, Hawthorn Woods, IL (US); Anatoly S. Belkin, Glenview, IL (US)

(21) Appl. No.: 18/663,654

(22) Filed: May 14, 2024

Related U.S. Application Data
(63) Continuation of application No. 18/102,047, filed on Jan. 26, 2023, now Pat. No. 11,986,623.
Application No. 18/102,047 is a continuation of application No. 16/835,027, filed on Mar. 30, 2020, now Pat. No. 11,571,508.
Application No. 16/835,027 is a continuation of application No. 14/472,549, filed on Aug. 29, 2014, now abandoned.
(60) Provisional application No. 61/872,333, filed on Aug. 30, 2013.

(51) Int. Cl. A61M 5/142; G06Q 30/018; G16H 20/17; G16H 40/63; G16H 40/67

(52) U.S. Cl. CPC A61M 5/142; G06Q 30/018; G16H 20/17; G16H 40/63; G16H 40/67; A61M 2205/18; A61M 2205/3553; A61M 2205/52

(57) ABSTRACT

A method and system for monitoring and managing a remote infusion regimen where a prescribed infusion regimen is provided and compared to subsequent infusion event data to determine if the data falls within predetermined parameters. If the data does not fall within predetermined parameters a monitoring technician, supportive caregiver, patient or some combination thereof are immediately notified so that corrective action may be taken.

### BACKGROUND OF THE INVENTION

### Field of the Invention

This invention is directed to a system and method that automates compliance of use of an infusion pump and more particularly a system and method that increases the probability that a prescribed infusion regimen is followed.

Ambulatory pumps are used on patients that can actively ambulate, which presents unique challenges in the care and management of patients. Within a healthcare setting, patients on ambulatory pumps remain under observation which limits noncompliance opportunities. In the home or other alternate site locations it becomes difficult to monitor and enforce patient compliance with prescribed infusion protocols. Home infusion patients generally prefer to be home versus the hospital in many cases, but can become non-compliant with infusion plans due to the technical challenges of managing infusions, a desire to skip or terminate treatments, or inadvertent misses.

Compliance with prescribed infusion plans is particularly important in the home setting as there is typically an expense associated with incorrectly followed infusion schedules. Antibiotics, the most ubiquitous home infusion medication class, are typically effective only if completed in a full protocol. The patient who feels better on day three or four and subsequently fails to complete a planned seven day infusion regimen will likely require a hospital re-admission, often through the Emergency Room, thereby negating the financial benefits and patient preference benefits of out-of-hospital infusion strategies. Such non-compliance becomes noteworthy not only to the patient but to the hospital as well as the payer/insurer, who generally supports alternate care strategies in order to avoid or truncate hospitalizations. Other medications delivered at home include medical necessities such as TPN, protein-derived therapeutics (which are typically very expensive), and oncolytics. All of these medications' effectiveness are dictated by timely and complete compliance. Payers generally will continue to be very invested in paying for home infusions that are in fact correctly administered. Further, discharging hospitals are growing more invested in post-discharge patient outcomes through pay-for-performance incentives and re-admission metrics are growing in importance through accountable care evolution.

In the past, compliance with home infusion plans has been loosely monitored through phone calls to patients or caregivers to ask about compliance. Further, compliance has been indirectly monitored through observation of appropriate re-order needs for IV medications and infusion supplies. Also, home nursing visits could be used to apply either of the two above techniques as well as ascertain probability of compliance based on patient condition. Finally, pump infusion and event history downloads can be used to note likely compliance. These techniques provide some guidance but do not offer a full perspective on patient compliance as phone inquiries can be misleading, medication inventory can be manipulated, home nurse visits may be infrequent, and pump activity downloads provide ex-post facto compliance datum but certainly are not timely enough to be actively useful. Therefore, a need exists in the art for a system and method that addresses these deficiencies.

An objective of the present invention is to provide a system and method that fully automates compliance monitoring and management of an infusion pump regimen.

Another objective of the present invention is to provide a system and method to link a prescribed infusion regiment to what is actually carried out by the home patient.

A still further objective of the present invention is to provide a system and method that increases the probability that a patient follows a prescribed infusion regimen.

Yet another objective of the present invention is to provide a system and method that increases the probability that an incomplete or incorrect regimen is addressed early in the non-compliance cycle.

Still, another objective of the present invention is to provide a system and method that captures value for a patient, a payer, an alternate site caregiver, and a discharging hospital.

One more objective of the present invention is to provide a system and method that increases motivation to use pumps for home infusions, whereby pumps can generally provide a safer means of controlled medication infusion than gravity infusions and provide a means by which to electronically capture the medication delivery details.

These and other objectives will be apparent to one of ordinary skill in the art based upon the following written description, drawings, and claims.

### SUMMARY OF THE INVENTION

A system and method for monitoring and managing remote compliance with an infusion regimen includes a healthcare provider having a controller with a processing unit, a database and a display. The database includes a plurality of patient records that include prescribed infusion regimens and patient outcome data. A patient is provided medication, an infusion pump, and one or more infusion sets in order to perform an infusion event at a remote location. Upon completing an infusion event, infusion event data is captured and transmitted to the healthcare provider where the data is stored in the patient outcome data and compared to the prescribed infusion regimen to determine if the data is within predetermined parameters. Notification is then provided to a monitoring technician and the patient.

Further, the anticipated infusion schedule is automatically monitored in order to trigger messaging or notification to the healthcare provider and/or patient in the event that appropriate infusion confirmation is not provided to match the infusion schedule within an appropriate time window.

Alternatively, the prescribed infusion regimen is downloaded to the infusion pump. The infusion event data from an infusion event is captured and compared with anticipatory infusion event data within the pump. If successful, the patient and/or supportive caregiver are provided with a code which is transmitted to the healthcare provider. If not within predetermined parameters an alarm or other notification is provided to the patient and/or supportive caregiver. This alarm or notification can be transmitted automatically through cellular communications, internet communications, or other means.

Alternatively, the prescribed infusion regimen is downloaded to the infusion pump. The infusion event data from an infusion event is captured and compared with anticipatory infusion event data within the pump. If the infusion schedule is not followed, the patient and/or supportive caregiver are messaged to initiate the infusion or to facilitate communication between the patient and caregiver.

### BRIEF DESCRIPTION OF THE DRAWINGS

FIG. 1 is a schematic view of a system for managing and monitoring a remote infusion regimen;

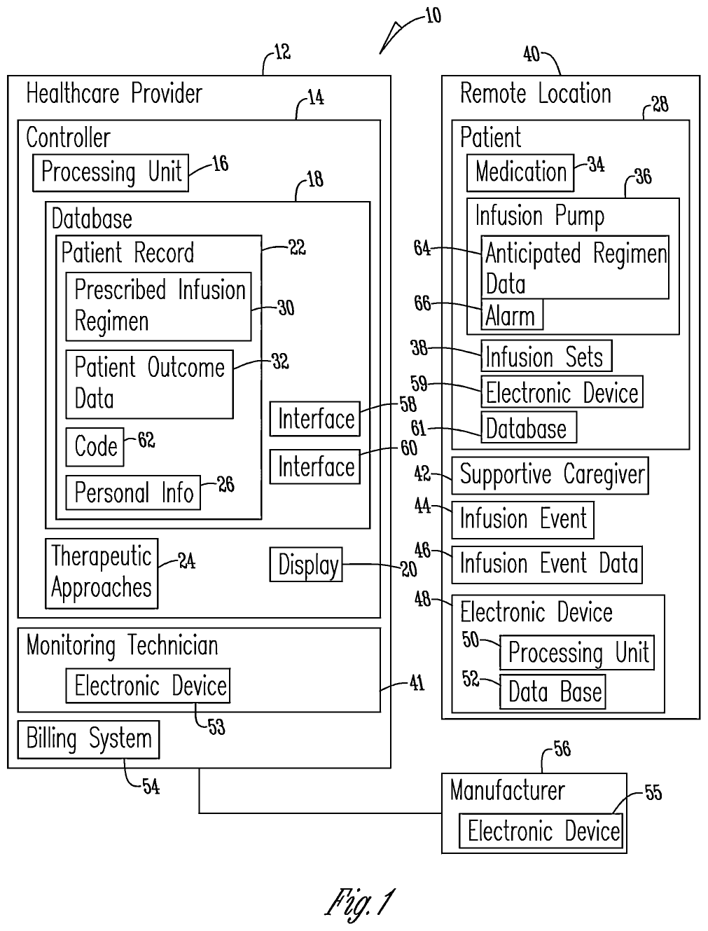

FIG. 2 is a flow diagram of a method for managing and monitoring a remote infusion regimen; and

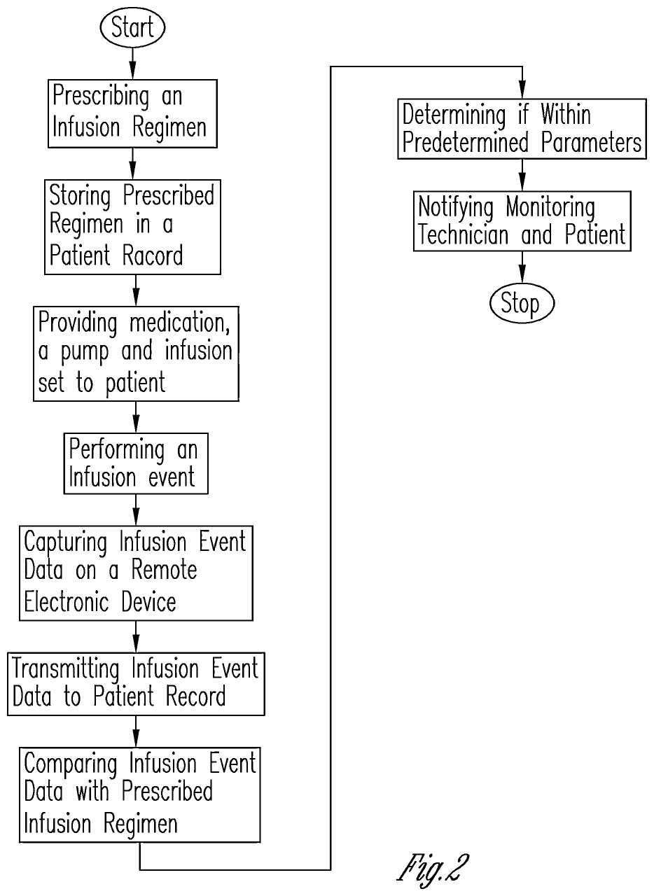

FIG. 3 is a flow diagram of a method for managing and monitoring a remote infusion regimen.

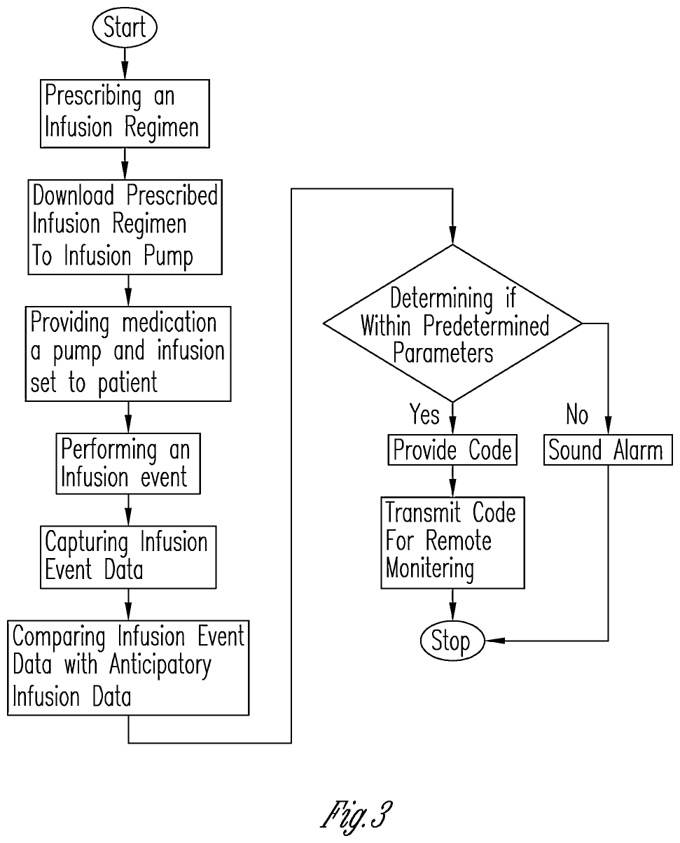

### DETAILED DESCRIPTION OF THE PREFERRED EMBODIMENTS

Referring to the Figures a system and method of monitoring and managing a remote infusion regimen 10 includes a monitoring healthcare provider 12 such as a discharging hospital, having a controller 14 with a processing unit 16, a database 18, and a display 20. The database 18 includes a plurality of patient records 22 and therapeutic approaches 24. Included in the patient record 22 is personal information 26 related to the patient 28, an infusion regimen 30 that has been prescribed by the healthcare facility 12, and patient outcome data 32.

Once the patient 28 has been prescribed an infusion regimen 30, which may consist of one or many infusions, the patient 28 is discharged from the healthcare facility 12. Medications 34, an infusion pump 36, and one or more pump tubing infusion sets 38 are provided to the patient 28 for remote use in the patient's home or other remote location 40. A monitoring clinician 41 such as a nurse, case manager or similar healthcare technician visits the remote location to train infusion techniques to the patient and/or a supportive caregiver 42 which includes a friend, a family member, or other individual who will initiate an infusion event 44. With each infusion event 44, infusion event data 46 is captured by an electronic device 48 connected to the infusion pump 36. Infusion event data includes, but is not limited to, start time, end time, medication, flow rate events, alarms, and the like. Infusion event data 46 may also include infusion set disconnections from the bag or patient. The electronic device 48 includes a cellular telephone, Wi-Fi, cable, modem link or the like and any combination of the above. In one embodiment, the electronic device 48 includes a processing unit 50, and a database 52. The electronic device may be separate from the infusion pumps or could be integrated within the infusion pump.

Once the infusion event data 46 is captured in real time, the data 46 is transmitted from the electronic device 48, via an electronic network, to the controller 14, where the data 46 is stored in the patient outcome data 32 in the patient record 22. The infusion event data 46 may also be stored on the database 52 of electronic device 48 or transferred to the patient record 22 in packets or through post infusion events.

The transmitted infusion event data 46 is compared by the processing unit 16 to the prescribed infusion regimen 30 to determine if the infusion event 44 is in compliance with the prescribed infusion regimen 30. The results of the comparison are stored in the patient record 22 and shown on display 20. The display 20 may show information related to a single patient record 22, or may provide an on-screen triage of multiple home patients for a dashboard-type snapshot of infusion activity.

In addition, if no infusion event data 46 is transmitted within a predetermined time period, this information is recorded in the patient record 22 and shown on display 22.

If the comparison of the infusion event data 46 with the prescribed infusion regimen 30 is not within pre-established parameters or if no infusion data 46 has been transmitted within a predetermined time period, the monitoring technician 41 is notified automatically by the controller 14 either through the display 20, or a communication to the monitoring technician's electronic device 53 such as by e-mail or text message. In addition, the patient 28 and/or supportive caregiver 42 may also be notified by the controller 14 via the electronic network by a communication to the electronic device 48. Further, the prescribed infusion regimen would be monitored automatically to identify missed infusions or incomplete infusions (i.e., those started but not successfully completed) or highly problematic infusions consisting of numerous alarms or inconsistencies. Upon notification, a skipped infusion or truncated regimen is immediately known, providing the opportunity to notify the patient 28, send out a nurse 41, or respond in another manner.

In addition to notification, the controller automatically generates a report on patient compliance against an approved prescribed regimen or clinical pathway for the payer, hospital, caregiver, and physician. The transmitted infusion event data 46 may also be tied to a billing system 54 for the healthcare provider 12 to ensure charge capture, and may be linked to the electronic device 55 of a manufacturer 56 to allow for development of improved pump usage statistics. Also, the patient record 22 has an interface 58 to capture orders from computerized physician order entry (CPOE) and/or pharmacy systems and an interface 60 for the electronic medical record (EMR) to log therapies.

In an alternative embodiment, the prescribed infusion regimen 30 is downloaded and stored in an electronic device 59 that includes a communication engine, wireless card or the like that is within the infusion pump 36 and the monitoring technician 41 is provided remote access through an electronic network to the infusion event data 46 that is stored on a database 61 within pump 36. Also, the pump would be provided with one or more predetermined codes 62 that are provided to the patient 28 and/or the supportive caregiver 42 upon completion of a successful infusion event 44 which is then provided to the monitoring technician 41 either through the electronic network such as by e-mail or through a telephone call. The monitoring technician has access to the predetermined code 62 which is stored in the patient record 22 and can monitor the remote infusion event 44 when the code is received.

In addition, the infusion event data 46 is compared to anticipated infusion data 64 that is stored on the infusion pump 36, with subsequent infusion event data 46 download or exception reporting to the patient record 22 or other caregiver-accessible database 52. When the infusion event data is not within preestablished parameters of prescribed or anticipated infusion regimens an on-pump alarm 66 is activated. Thus, patients 28 are notified immediately if they are not performing infusions within the allotted time, at appropriate start times, or the like.

Accordingly, a method and system of monitoring and managing the compliance of a remote infusion regimen has been disclosed that at the very least, meets the stated objectives.

What is claimed is:

1. A system for monitoring and managing remote compliance with an infusion regimen, comprising:
a controller having a processor and database that are connected to an electronic network;
a patient record stored in the database having a prescribed infusion regimen;
an infusion pump located remotely to the controller and connected to an electronic device that transmits infusion event data via the electronic network to the controller where the transmitted data is stored and compared to the prescribed infusion regimen.

2. The system of claim 1 further comprising a notification that is transmitted to at least one of a supportive caregiver, a monitoring nurse, or a technician when the transmitted infusion event data is not within predetermined compliance requirements.

3. The system of claim 1 wherein the electronic device has a database that stores the infusion event data.

4. The system of claim 1 wherein the database includes patient outcome information that is linked to therapeutic approaches.

5. The system of claim 1 wherein transmission of infusion event data is linked to a billing system to ensure charge capture.

6. A method of monitoring and managing remote compliance with an infusion regiment, comprising the steps of:
prescribing a remote infusion regimen;
storing the prescribed remote infusion regimen in a patient record of a database of a controller having a processing unit and a display;
capturing data on a remote electronic device of an infusion event for a patient using an infusion pump;
transmitting and then storing the captured infusion event data to the database of the controller via an electronic network;
comparing the transmitted infusion event data with the prescribed infusion regimen; and
transmitting a notification to a monitoring caregiver based upon the comparison.

7. The method of claim 6 further comprising the step of transmitting a notification to a supportive caregiver from the controller via the electronic network when the transmitted infusion data is not compliant with the prescribed infusion regimen.

8. The method of claim 6 further comprising the step of transmitting the infusion event data to a database of an infusion pump manufacturer via the electronic network to allow development of improved pump usage statistics.

9. The method of claim 6 wherein the captured infusion event data is stored in a database on the electronic device of the supportive caregiver.

10. A system for monitoring and managing remote compliance with an infusion regimen, comprising:
an infusion pump having a processor and a database wherein the database includes a prescribed infusion regimen and stores infusion event data;
an electronic device disposed within an infusion pump and connected to an electronic network;
a controller having a processing unit, a database, and a display that receives infusion event data from the electronic device via the electronic network and compares the transmitted event data with the prescribed infusion regimen to determine compliance.

11. The system of claim 10 wherein the controller provides a notification to a monitoring caregiver when the infusion event data is not in compliance.

12. The system of claim 10 wherein the infusion pump provides a code to a patient on completion of an infusion event which the patient provides to a monitoring caregiver following a successful infusion event.

13. The system of claim 10 wherein the controller on the infusion pump compares infusion event data with predetermined anticipated data to determine compliance.

14. The system of claim 10 having a pharmacy system interface to capture orders.

15. The system of claim 10 wherein the monitoring caregiver display provides on-screen triage of multiple remote infusion activity.
